(19) United States
(12) Patent Application Publication
(10) Pub. No.: US 2021/0349103 A1
(43) Pub. Date: Nov. 11, 2021

(54) METABOLIC BIOMARKERS OF NAFLD/NASH AND RELATED DISEASE PHENOTYPES AND METHODS OF USING SAME

(71) Applicant: Duke University, Durham, NC (US)

(72) Inventors: Christopher B. NEWGARD, Durham, NC (US); Phillip WHITE, Durham, NC (US); Thomas GRENIER-LAROUCHE, Durham, NC (US)

(21) Appl. No.: 17/254,118

(22) PCT Filed: Jun. 18, 2019
(86) PCT No.: PCT/US19/37746

§ 371 Date: Dec. 18, 2020

Related U.S. Application Data
(60) Provisional application No. 62/686,154, filed on Jun. 18, 2018.

(51) Int. Cl. G01N 33/68

(52) U.S. Cl. CPC G01N 33/6848; G01N 33/6893

(57) ABSTRACT

The present invention relates to biomarkers and methods of using the same. Specifically, the present invention relates to biomarkers for nonalcoholic fatty liver disease and related disease phenotypes and methods of using the same.

### PRIORITY

This application claims priority to U.S. Provisional Application No. 62/686,154, filed Jun. 18, 2018, the entire contents of which are incorporated herein by reference.

### FEDERAL FUNDING

This invention was made with government support under Federal Grant No. P01-DK58398 awarded by the NIH. The government has certain rights in the invention.

### TECHNICAL FIELD

The present invention relates to biomarkers and methods of using the same. Specifically, the present invention relates to biomarkers for nonalcoholic fatty liver disease and related disease phenotypes and methods of using the same.

### BACKGROUND

Non-alcoholic fatty liver disease (NAFLD) is characterized by neutral lipid accumulation in the liver. NAFLD encompasses a histologic spectrum ranging from isolated hepatic steatosis to nonalcoholic steatohepatitis (NASH) characterized by lipid accumulation, inflammation, hepatocyte ballooning, and varying degrees of fibrosis. This more pathogenic form of NAFLD progresses to fibrosis in approximately 35% of patients, significantly raising the risk for development of hepatocellular carcinoma (HCC), cirrhosis, and acute liver failure. Advanced NAFLD is also a significant risk factor for development of type 2 diabetes and cardiovascular diseases (CVD). The severity of hepatic fibrosis is the primary predictor of increased morbidity and mortality in patients with NAFLD.

The prevalence of nonalcoholic fatty liver disease (NAFLD) continues to increase with the growing obesity epidemic. The obesity pandemic has driven a sharp increase in the incidence of NAFLD in recent years to an estimated incidence in the United States of 25%. NALFD-related liver failure is now comparable to hepatitis C as a primary cause of liver transplants in the United States. Coincidentally, the rising tide of NAFLD has also lowered the quality of the available liver donor pool.

A major limitation for intervention in this disease is the absence of plasma biomarkers identifying NAFLD and its progression to NASH. Although there is increasing public awareness for the risk of liver disease progression in patients with NASH, stratifying patients “at risk” for advanced hepatic fibrosis, and thus associated negative clinical outcomes, is hindered by the need for liver biopsy and the lack of non-invasive biomarkers. Other existing imaging methods such as magnetic resonance imaging (MRI) are expensive and significantly less effective at discriminating NAFLD status in persons with obesity. Accordingly, non-invasive biomarkers for identifying and/or staging the progression of NAFLD are needed to enable clinicians to stratify risk and guide appropriate care and management of the disease.

### SUMMARY

In some aspects, provided herein are methods of diagnosing and treating non-alcoholic fatty liver disease (NAFLD) in a subject. In some embodiments, the methods include obtaining a sample from the subject, measuring the concentration of one or more branched chain keto-acids (BCKAs) in the sample, diagnosing the patient with NAFLD when the concentration of the one or more BCKAs is elevated compared to a reference value, and providing therapy to the subject diagnosed with NAFLD.

In other embodiments, the methods of diagnosing and treating NAFLD include obtaining a sample from the subject, measuring the concentration of one or more branched chain keto-acids (BCKAs) in the sample, measuring the concentration of one or more branched chain amino acids (BCAAs) in the sample, determining a ratio of one or more BCKAs: one or more BCAAs in the sample, diagnosing the patient with NAFLD when the ratio of one or more BCKAs: one or more BCAAs is elevated compared to a reference value, and providing therapy to the subject diagnosed with NAFLD.

In other embodiments the methods of diagnosing and treating NAFLD include obtaining a sample from the subject, measuring a concentration of one or more branched chain keto-acids (BCKAs) in the sample, measuring a concentration of one or more branched chain amino acids (BCAAs) in the sample, determining a ratio of one or more BCKAs: one or more BCAAs in the sample, diagnosing the patient with NAFLD when the concentration of the one or more BCKAs and the ratio of the one or more BCKAs: the one or more BCAAs is elevated compared to a reference value, and providing therapy to the subject diagnosed with NAFLD.

In other aspects, provided herein are biomarker panels. In some embodiments, the panel of biomarkers includes one or more branched chain keto-acids (BCKAs) and one or more branched chain amino-acids (BCAAs).

In other aspects, provided herein are methods including steps of obtaining a sample from the subject, measuring a concentration of one or more branched chain keto-acids (BCKAs) and one or more branched chain amino acids (BCAAs) in the sample, and calculating the ratio of one or more BCAAs: one or more BCAAs: in the sample.

In accordance with any of the aspects and embodiments described herein, the BCKAs may be any one of more of alpha-ketoisovalereric acid (KIV), alpha-ketoisocaproic acid (KIC), and alpha-keto-beta-methylvaleric acid (KMV) and BCAAs may be any one of more of leucine, isoleucine, and valine.

In accordance with any of the aspects and embodiments described herein, suitable therapies for NAFLD include antioxidants, cytoprotective agents, antidiabetic agents, insulin-sensitizing agents, anti-hyperlipidemic agents, acetyl co-A carboxylase inhibitors, and ATP-citrate lyase inhibitors.

In accordance with any of the aspects and embodiments described herein, the subject may be human. In some embodiments, the subject may be obese or overweight. In some embodiments, the subject may be female. In certain embodiments, the subject may express the Ile148Met variant of the PNPLA3 gene.

### BRIEF DESCRIPTION OF THE DRAWINGS

FIG. 1. Association of plasma KIV with features of NAFLD/NASH in women carrying the major PNPLA3 allele. (A) Association of plasma KIV with steatosis grade and presence of NASH. (B) Association of plasma KIV with ballooning and lobular inflammation.

FIG. 2. Genotype and sex interactions. (A) Association of Factor 10 with NASH status not present in carriers of the G allele of PNPLA3. (B) Association of BCKA:BCAA with steatosis grade is present in females but not males.

### DETAILED DESCRIPTION

The present disclosure is predicated, at least in part, on the discovery of non-invasive biomarkers for the diagnosis of non-alcoholic fatty liver disease in a subject.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### Definitions

Unless otherwise defined, all technical terms used herein have the same meaning as commonly understood by one of ordinary skill in the art to which this disclosure belongs. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The use of the terms “a” and “an” and “the” and “at least one” and similar referents in the context of describing the invention (especially in the context of the following claims) are to be construed to cover both the singular and the plural, unless otherwise indicated herein or clearly contradicted by context.

The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context.

As used herein, the term “about” is used to provide flexibility to a numerical range endpoint by providing that a given value may be “slightly above” or “slightly below” the endpoint without affecting the desired result. In some embodiments, “about” may refer to variations of in some embodiments ±20%, in some embodiments ±10%, in some embodiments ±5%, in some embodiments ±1%, in some embodiments ±0.5%, and in some embodiments ±0.1% from the specified amount.

As used herein, the terms “comprise”, “include”, and linguistic variations thereof denote the presence of recited feature(s), element(s), method step(s), etc. without the exclusion of the presence of additional feature(s), element(s), method step(s), etc.

Recitation of ranges of values herein are merely intended to serve as a shorthand method of referring individually to each separate value falling within the range, unless otherwise-indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

The term “amino acid analog” refers to a natural or unnatural amino acid where one or more of the C-terminal carboxy group, the N-terminal amino group and side-chain bioactive group has been chemically blocked, reversibly or irreversibly, or otherwise modified to another bioactive group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

As used herein, the term “biomarker” refers to a naturally occurring biological molecule present in a subject at varying concentrations useful in predicting the risk, incidence, or severity of a disease or a condition, such as NAFLD or other related disease phenotypes. For example, the biomarker can be a protein, acylcarnitines, amino acids, branched chain keto-acids, and/or other conventional metabolites that present in higher or lower amounts in a subject at risk for, or suffering from, NAFLD or related disease phenotypes. In some embodiments, the biomarker is a protein. A biomarker may also comprise any naturally or non-naturally occurring polymorphism (e.g., single-nucleotide polymorphism [SNP]) present in a subject that is useful in predicting the risk or incidence of NAFLD. In some embodiments, the biomarker one or more branched chain keto-acids (BCKAs) and one or more branched chain amino-acids (BCAAs). In some embodiments, measurement of the one or more biomarkers may be used to calculate a ratio of one or more BCKAs: one or more BCAAs, which may also be indicative of the presence of severity of NAFLD in a subject.

As used herein, the term “branched” refers to a central carbon atom bound to three or more carbon atoms.

As used herein, the terms “branched chain amino acid” or “BCAA” are used interchangeably herein to refer to an amino acid having an aliphatic side chain and a branch. Exemplary branched chain amino acids include leucine, isoleucine, and valine.

As used herein, the terms “branched chain keto-acid”, or “BCKA” are used interchangeably herein. A branched chain keto-acid refers to a keto acid containing a carboxylic acid group and a ketone group and a branch (e.g. a central carbon atom bound to three or more carbon atoms). Branched chain keto-acids may be branched chain alpha-keto acids. Branched chain ketoacids may be branched chain beta-keto acids. Branched chain keto-acids may be branched chain gamma-keto acids. A BCKA may be a metabolite in one or more BCAA synthesis or BCAA catabolism pathways. For example, a branched chain keto-acid may be a metabolite produced during synthesis and/or catabolism of valine, leucine, or isoleucine. Exemplary branched chain keto-acids include alpha-ketoisovalereric acid (e.g. alpha-ketoisovalerate) (KIV), alpha-ketoisocaproic acid (KIC), and alpha-keto-beta-methylvaleric acid (KMV).

As used herein, the terms “co-administration” and variations thereof refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Accordingly, co-administration may be especially desirable in embodiments where the co-administration of two or more agents results in sensitization of a subject to beneficial effects of one of the agents via co-administration of the other agent.

The term “carrier” as used herein refers to any pharmaceutically acceptable solvent of agents that will allow a therapeutic composition to be administered to the subject. A “carrier” as used herein, therefore, refers to such solvent as, but not limited to, water, saline, physiological saline, oil-water emulsions, gels, or any other solvent or combination of solvents and compounds known to one of skill in the art that is pharmaceutically and physiologically acceptable to the recipient human or animal.

The term “control subject” is used herein to refer to a subject not presently experiencing or diagnosed with NAFLD or related disease phenotypes.

As used herein, the terms “effective amount” or “therapeutically effective amount” are used interchangeably herein to refer to an amount sufficient to effect beneficial or desirable biological and/or clinical results.

As used herein, the term “fibrosis” refers to the formation of scar tissue in the liver. The term “fibrosis” may refer to “cirrhosis”, which is used herein to denote late-stage (e.g. advanced) fibrosis in the liver.

As used herein, the terms “non-alcoholic fatty liver disease” and “NAFLD” are used interchangeably to refer to a range of conditions affecting people who drink little to no alcohol characterized, at least in part, by excess fat stored in liver cells (e.g. steatosis). NAFLD may be characterized by any combination of features including steatosis, fibrosis, enlarged liver, fatigue, abdominal pain, abdominal swelling, enlarged blood vessels, enlarged breasts, enlarged spleen, red palms, and jaundice. NAFLD refers to a spectrum of conditions that may range in severity or degree, depending on the progression of the disease in a given individual. In some embodiments, non-alcoholic liver disease may refer to non-alcoholic steatohepatitis (“NASH”), a more severe form of NAFLD characterized by characterized by lipid accumulation, inflammation, hepatocyte ballooning, and varying degrees of fibrosis in the liver.

As used herein, the term “pharmaceutical composition” refers to the combination of an active agent with a carrier, inert or active, making the composition especially suitable for therapeutic use.

The term “pharmaceutically acceptable” as used herein refers to a compound or composition that will not impair the physiology of the recipient human or animal to the extent that the viability of the recipient is compromised. For example, “pharmaceutically acceptable” may refer to a compound or composition that does not substantially produce adverse reactions, e.g., toxic, allergic, or immunological reactions, when administered to a subject.

As used herein, the terms “prevent,” “prevention,” and preventing” may refer to reducing the likelihood of a particular condition or disease state (e.g., non-alcoholic steatohepatitis) from occurring in a subject not presently experiencing or afflicted with the condition or disease state. The terms do not necessarily indicate complete or absolute prevention. For example “preventing NASH” refers to reducing the likelihood of NASH occurring in a subject not presently experiencing or diagnosed with NASH. For example, preventing NASH may reduce the likelihood of NASH occurring in a subject currently diagnosed with mild NAFLD but not currently diagnosed with NASH. The terms may also refer to delaying the onset of a particular condition or disease state (e.g., NASH) in a subject not presently experiencing or afflicted with the condition or disease state. In order to “prevent” a condition, a composition or method need only reduce the likelihood and/or delay the onset of the condition, not completely block any possibility thereof “Prevention,” encompasses any administration or application of a therapeutic or technique to reduce the likelihood or delay the onset of a disease developing (e.g., in a mammal, including a human). Such a likelihood may be assessed for a population or for an individual.

The term “reference” and “reference value” are used interchangeably herein to refer to a value associated with a control subject (e.g. a subject not currently diagnosed with or experiencing NAFLD or a related disease state). For example, a reference value may refer to a value corresponding to the concentration of one or more BCKAs, one or more BCAAs, and/or the ratio of one or more BCKAs: one or more BCAAs. A reference value may be obtained prior to measuring the desired value in the sample obtained from the subject. A reference value may be obtained after measuring the desired value in the sample obtained from the subject. A reference value may be obtained from a sample collected from a control subject or may be a range of acceptable values based upon a plurality of samples collected from one or more control subjects. For example, a reference value may be a range of values obtained from a known database of values in control subjects.

The terms “sample” or “biological sample” as used interchangeably herein includes any suitable sample isolated from the subject or from a control subject. Suitable samples include, but are not limited to, a sample containing tissues, cells, and/or biological fluids isolated from a subject. Examples of samples include, but are not limited to, tissues, cells, biopsies, blood, lymph, serum, plasma, urine, saliva, mucus and tears. In one embodiment, the sample comprises a serum sample, a blood sample, or a plasma sample. A sample may be obtained directly from a subject or a control (e.g., by blood or tissue sampling) or from a third party (e.g., received from an intermediary, such as a healthcare provider or lab technician).

As used herein, the term “steatosis” refers to the accumulation of fat in the cells of the liver.

As used herein, the terms “subject” and “patient” are used interchangeably herein and refer to both human and nonhuman animals. The term “nonhuman animals” includes all vertebrates, e.g., mammals and non-mammals, such as nonhuman primates, sheep, dogs, cats, horses, cows, chickens, amphibians, reptiles, and the like. In some embodiments, the subject is a human. In some embodiments, the subject is a human. In particular embodiments, the subject may be overweight or obese. In particular embodiments, the subject may be male. In other embodiments, the subject may be female. In certain embodiments, the subject expresses the Ile148Met variant of PNPLA3. In certain embodiments, the subject is a human suffering from, or is at risk of suffering from, NAFLD or related disease phenotypes.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. In some embodiments, treating NAFLD refers to the management and care of the subject for combating and reducing NAFLD. Treating NAFLD may reduce, inhibit, ameliorate and/or improve the onset of the symptoms or complications, alleviating the symptoms or complications of the disease, or eliminating the disease. As used herein, the term “treatment” is not necessarily meant to imply cure or complete abolition of the liver disease. Treatment may refer to the inhibiting or slowing of the progression of NAFLD or related disease phenotypes, reducing the incidence of NAFLD or related disease phenotypes, or preventing additional progression of NAFLD or related disease phenotypes. For example, treatment may refer to stopping the progression of NAFLD characterized by isolated steatosis to the more severe form of NAFLD, referred to herein as NASH.

### Biomarkers and Methods

In one aspect, described herein is a panel of biomarkers. The panel comprises one or more branched chain keto-acids (BCKAs) and one or more branched chain amino-acids (BCAAs). The panel may comprise any suitable one or more BCKAs and any suitable one or more BCAAs. For example, the branched chain keto-acids may be selected from alpha-ketoisovalereric acid (KIV), alpha-ketoisocaproic acid (KIC), and alpha-keto-beta-methylvaleric acid (KMV) and the branched chain amino acids may be selected from leucine, isoleucine, and valine. In some embodiments, the panel comprises one BCKA and one BCAA. For example, the panel may comprise KIV and valine. As another example, the panel may comprise KIC and leucine. As another example, the panel may comprise KMV and isoleucine.

In other embodiments, the panel may comprise more than one BCKA and more than one BCAA. For example, the panel may comprise two BCKAs and two BCAAs. As another example, the panel may comprise three BCKAs and three BCAAs. For example, the panel may comprise KIV, KIC, and KMV and leucine, isoleucine, and valine.

In some embodiments, the panels described herein may be used in a method of diagnosing NAFLD in a subject. In such embodiments, an elevated concentration of one or more BCKAs in the panel compared to a control and/or an elevated ratio of one or more BCKAs: BCAAs in the panel compared to a control is positively correlated with NAFLD in the subject. For example, an elevated concentration of KIV may be correlated with NAFLD in the subject. As another example, an elevated concentration of KIV and an elevated ratio of KIV, KIC, and KMV:leucine, isoleucine, and valine may be correlated with NAFLD in the subject.

In another aspect, provided herein are methods of diagnosing and treating NAFLD in a subject. The methods comprise obtaining a sample from the subject. In some embodiments, the method comprises measuring the concentration of one or more BCKAs in the sample. Any suitable BCKA or combination of BCKAs may be measured. In some embodiments, the concentration of one BCKA in the subject may be measured. In other embodiments, the concentration of more than one BCKA in the subject may be measured. Suitable BCKAs include, but are not limited to, ketoisovalereric acid (KIV), ketoisocaproic acid (KIC), and ketomethylvaleric acid (KMV). For example, the method may comprise measuring the concentration of KIV in the sample. The method may comprise measuring the concentration of KIC in the sample. The method may comprise measuring the concentration of KMV in the sample. In some embodiments, the method may comprise measuring the concentration of KIV, KIC, and KMV in the sample.

In some embodiments, the method comprises measuring the concentration of one or more BCAAs in the sample. The concentration of any suitable BCAA or combination of BCAAs may be measured. In some embodiments, the concentration of one BCAA may be measured. In some embodiments, the concentration of more than one BCAA may be measured. For example, the concentration of any one or more of leucine, isoleucine, or valine may be measured. In some embodiments, the concentration of leucine may be measured. In some embodiments, the concentration of isoleucine may be measured. In other embodiments, the concentration of leucine, isoleucine, and valine may be measured.

In some embodiments, the method comprises measuring the concentration of one or more BCKAs and the concentration of one or more BCAAs in the subject. In accordance with such embodiments, the method may further comprise calculating a ratio of the one or more BCKAs: the one or more BCAAs in the sample. Any one or more BCKAs and any one or more BCAAs may be used. In some embodiments, the method may comprise calculating a ratio of one BCKA:one BCAA in the sample. For example, the method may comprise measuring the concentration KIV in the sample, measuring the concentration of valine in the sample, and calculating the ratio of KIV:valine in the sample.

In other embodiments, the method may comprise calculating a ratio of more than one BCKA:more than one BCAA in the sample. For example, the method may comprise measuring the concentration of KIV and KMC in the sample, measuring the concentration of valine and isoleucine in the sample, and calculating the ratio of the concentration of KIV and KMV: the concentration of valine and isoleucine in the sample. In some embodiments, the method may comprise measuring the ratio of total BCKA concentration:total BCAA concentration in the sample. For example, the method may comprise measuring the concentration of KIV, KMV, and KIC in the sample, measuring the concentration of leucine, isoleucine, and valine in the sample, and calculating the ratio of the concentration of KIV, KMV, and KIC in the sample: the concentration of leucine, isoleucine, and valine in the sample. The above examples are not intended to be limiting in any way, it is understood that the concentration of any one or more BCKAs and the concentration of any one or more BCAAs may be measured and used in calculating the desired ratio.

In accordance with any of the above described embodiments, the method further comprises comparing the concentration of the one or more BCKAs, the concentration of one or more BCAAs, and/or the calculated ratio of one or more BCKAs:one or more BCAAs in the sample to reference value. The methods further comprise diagnosing the subject with NAFLD when the concentration of the one or more BCKAs and/or the calculated ratio of the one or more BCKAs: the one or more BCAAs is elevated compared to the reference value. For example, the method may comprise diagnosing the subject with NAFLD when the concentration of the one or more BCKAs is elevated compared to a reference value. For example, the method may comprise measuring KIV and diagnosing the subject with NAFLD when the concentration of KIV is elevated compared to a reference value.

Alternatively or in addition, the method may comprise diagnosing the subject with NAFLD when the ratio of the one or more BCKAs: the one or more BCAAs is elevated compared to a control. For example, the method may comprise diagnosing the subject with NAFLD when the ratio of KIV, KIC, and KMV:isoleucine, leucine, and valine is elevated compared to a control. In other embodiments, the method comprises diagnosing the subject with NAFLD when the concentration of one or more BCKAs and the ratio of the one or more BCKAs: the one or more BCAAs is elevated compared to a control. For example, the method may comprise diagnosing the subject with NAFLD when the concentration of KIV and the ratio of ratio of KIV, KIC, and KMV:isoleucine, leucine, and valine is elevated compared to a control.

In another aspect, provided herein is a method comprising obtaining a sample from the subject, measuring the concentration of one or more branched chain keto-acids (BCKAs) and the concentration of one or more branched chain amino acids (BCAAs) in the sample; and calculating the ratio of one or more BCAAs:one or more BCAAs: in the sample. Any suitable one or more BCKAs and any suitable one more BCAAs may be used. In some embodiments, branched chain keto-acids are selected from alpha-ketoisovalereric acid (KIV), alpha-ketoisocaproic acid (KIC), and alpha-keto-beta-methylvaleric acid (KMV) and branched chain amino acids are selected from leucine, isoleucine, and valine. In some embodiments, an elevated concentration of the one or more BCKAs compared to a control sample and/or an elevated ratio of one or more BCKAs:one or more BCAAs in the sample is positively correlated with non-alcoholic fatty liver disease (NAFLD) in the subject. For example, an elevated concentration of KIV in the sample may be positively correlated with NAFLD in the subject. As another example, an elevated concentration of KIV in the sample and an elevated ratio of KIV, KIC, and KMV:isoleucine, leucine, and valine in the sample may be positively correlated with NAFLD in the subject.

In accordance with any of the embodiments described herein, the methods may further comprise measuring the concentration or activity of other suitable metabolites in combination with the one or more BCKAs and/or the one or more BCAAs. Other suitable metabolites include, for example, amino acids, acylcarnitines, ceramides, or other biomarkers that may be associated with fatty liver disease such as NAFLD and related disease phenotypes.

A positive correlation with NAFLD or a diagnosis of NAFLD may indicate that the subject is experiencing any one or more symptoms at any severity associated with NAFLD. For example, a diagnosis of NAFLD may indicate that the subject is afflicted with mild NAFLD, characterized by isolated hepatic steatosis. In other embodiments, a diagnosis of NAFLD may indicate that the subject is afflicted with non-alcoholic steatohepatitis. In such embodiments, a diagnosis of NASH may indicate that the subject is experiencing any one of more of steatosis, inflammation, hepatocyte ballooning, and varying degrees of fibrosis in the liver.

In accordance with any of the embodiments described herein, the concentration of the one or more BCKAs and/or the concentration of the one or more BCAAs may be measured by any suitable method or combination of methods as known in the art. Suitable methods include, but are not limited to, methods utilizing:mass spectrometry (MS), high performance liquid chromatography (HPLC), isocratic HPLC, gradient HPLC, normal phase chromatography, reverse phase HPLC, size exclusion chromatography, ion exchange chromatography, capillary electrophoresis, microfluidics, chromatography, gas chromatography (GC), thin-layer chromatography (TLC), immobilized metal ion affinity chromatography (IMAC), affinity chromatography, immunoassays, and/or colorimetric assays. In some embodiments, concentration of the one or more BCKAs and/or concentration of the one or more BCAAs is measured by mass spectrometry. Any suitable mass spectrometry method may be used. For example, suitable mass spectrometry methods include liquid chromatography mass spectrometry, gas chromatography mass spectrometry, capillary electrophoresis mass spectrometry, tandem mass spectrometry, and the like.

In accordance with any of the embodiments described herein, the method may further comprise providing treatment to the subject. For example, the method may further comprise providing treatment to the subject diagnosed with NAFLD. Any suitable treatment for NAFLD may be used. Suitable treatments include therapeutic agents and/or surgery. For example, treatment may comprise bariatric surgery. Alternatively or in addition, treatment may comprise one or more therapeutic agents. Suitable therapeutic agents include antioxidants, cytoprotective agents, antidiabetic agents, insulin-sensitizing agents, anti-hyperlipidemic agents, acetyl co-A carboxylase inhibitors, and ATP-citrate lyase inhibitors.

In accordance with any of the embodiments described herein, therapeutic agents may be administered by themselves or as a part of a pharmaceutical composition comprising the one or more therapeutic agents and one or more carriers. Suitable carriers depend on the intended route of administration to the subject. Contemplated routes of administration include those oral, rectal, nasal, topical (including transdermal, buccal and sublingual), vaginal, parenteral (including subcutaneous, intramuscular, intravenous and intradermal) and pulmonary administration. In some embodiments, the composition or compositions are conveniently presented in unit dosage form and are prepared by any method known in the art of pharmacy. Such methods include the step of bringing into association the active ingredient with the carrier which constitutes one or more accessory ingredients. In general, the formulations are prepared by uniformly and intimately bringing into association (e.g., mixing) the active ingredient with liquid carriers or finely divided solid carriers or both, and then if necessary shaping the product.

Formulations of the present disclosure suitable for oral administration may be presented as discrete units such as capsules, cachets or tablets, wherein each preferably contains a predetermined amount of the one or more therapeutic agents as a powder or granules; as a solution or suspension in an aqueous or non-aqueous liquid; or as an oil-in-water liquid emulsion or a water-in-oil liquid emulsion. In other embodiments, the composition is presented as a bolus, electuary, or paste, etc.

Preferred unit dosage formulations are those containing a daily dose or unit, daily subdose, or an appropriate fraction thereof, of an agent.

It should be understood that in addition to the ingredients particularly mentioned above, the compositions may include other agents conventional in the art having regard to the route of administration in question. For example, compositions suitable for oral administration may include such further agents as sweeteners, thickeners and flavoring agents. Still other formulations optionally include food additives (suitable sweeteners, flavorings, colorings, etc.), phytonutrients (e.g., flax seed oil), minerals (e.g., Ca, Fe, K, etc.), vitamins, and other acceptable compositions (e.g., conjugated linoelic acid), extenders, preservatives, and stabilizers, etc.

Various delivery systems are known and can be used to administer compositions described herein, e.g., encapsulation in liposomes, microparticles, microcapsules, receptor-mediated endocytosis, and the like. Methods of delivery include, but are not limited to, intra-arterial, intra-muscular, intravenous, intranasal, and oral routes. In specific embodiments, it may be desirable to administer the compositions of the disclosure locally to the area in need of treatment; this may be achieved by, for example, and not by way of limitation, local infusion during surgery, injection, or by means of a catheter.

Therapeutic amounts are empirically determined and vary with the pathology being treated, the subject being treated and the efficacy and toxicity of the agent. It is understood that therapeutically effective amounts vary based upon factors including the age, gender, and weight of the subject, among others. It also is intended that the compositions and methods of this disclosure be co-administered with other suitable compositions and therapies.

In general, suitable doses of the therapeutic agent may range from about 1 ng/kg to about 1 g/kg. For example, a suitable dose may be from about 1 ng/kg to about 1 g/kg, about 100 ng/kg to about 900 mg/kg, about 200 ng/kg to about 800 mg/kg, about 300 ng/kg to about 700 mg/kg, about 400 ng/kg to about 600 mg/kg, about 500 ng/kg to about 500 mg/kg, about 600 ng/kg to about 400 mg/kg, about 700 ng/kg to about 300 mg/kg, about 800 ng/kg to about 200 mg/kg, about 900 ng/kg to about 100 mg/kg, about 1 μg/kg to about 50 mg/kg, about 10 μg/kg to about 10 mg/kg, about 100 μg/kg to about 1 mg/kg, about 200 μg/kg to about 900 μg/kg, about 300 μg/kg to about 800 μg/kg, about 400 μg/kg to about 700 μg/kg, or about 500 μg/kg to about 600 μg/kg.

The one or more therapeutic agents may be administered to the subject at any desired frequency. For example, the one or therapeutic agents may be administered to the subject more than once per day (e.g. twice per day, three times per day, four times per day, and the like), once per day, once every other day, once a week, and the like. The one or more therapeutic agents may be provided to the subject for any desired duration. For example, the one or more therapeutic agents may be administered to the subject for at least one week, at least two weeks, at least three weeks, at least one month, at least two months, at least three months, at least six months, at least one year, at least two years, at least three years, at least four years, at least five years, at least ten years, at least twenty years, or for the lifetime of the subject.

In another aspect, provided herein is a kit for use in detecting one or more BCKAs and/or one or more BCAAs in a sample. For example, provided herein is a kit for detecting one or more BCKAs and one or more BCAAs in a sample. The kit may comprise a means for detecting the one or more BCKAs and/or the one or more BCAAs in a sample. For example, the kit may comprise a means for detecting one or more of KIV, KIC, and KMV in a sample. Alternatively or in combination, the kit may comprise a means for detecting one or more of leucine, isoleucine, and valine in a sample. The kit may comprise additional reagents necessary for detecting the one or more BCKAs and or the one or more BCAAs in the sample (e.g. tubes, plates, pipette tips, buffers, salts, pH balancing reagents, proteases, phosphatases, and the like).

The following examples further illustrate the invention but, of course, should not be construed as in any way limiting its scope.

### Example 1

This example evaluated whether circulating levels of BCKAs could be a sensitive marker of NAFLD status in persons with obesity. This hypothesis was tested in a cohort of 288 bariatric surgery patients with severe obesity (body mass index (BMI) >35 kg/m²) from the Quebec Heart and Lung Institute (QHLI) Biobank in whom liver biopsies were taken at the time of bariatric surgery for histological grading of steatosis, inflammation, ballooning, and fibrosis.

Materials and Methods

Study participants: The study population included 288 patients (191 women and 97 men) of European ancestry with severe obesity (BMI >35 kg/m²) from the eastern provinces of Canada that underwent bariatric surgery at the Québec Heart and Lung Institute (QHLI, Québec City, QC, Canada). 404 samples in the QHLI biobank met the initial inclusion criteria for the study which were:Hba1c<6% and FPG<7 mM, histologic NAFLD characterization, consent for genetic studies, and not on diabetes medications. NAFLD was present in 79% of these patients (steatosis grade 1: 55%, grade 2: 18%, grade 3: 6%). The 288 samples used for the study were selected in order to have a well-matched population for age, sex, BMI, and glucose tolerance spanning the range of steatosis grades (0-3) and steatosis with NASH (defined as the presence of steatosis alongside both lobular inflammation and ballooning) vs no NASH (see Table 1 for stratification of the population by steatosis grade and NASH status).

TABLE 1
Baseline characteristics of the study population.

| | Steatosis Grade | | | | | | | | |
|---|---|---|---|---|---|---|---|---|---|
| | Grade 0 | | Grade 1 | | Grade 2 | | Grade 3 | | p-value |
| n | 57 | | 118 | | 58 | | 55 | | |
| Age | 40.8 | (10.1) | 41.8 | (9.9) | 41.1 | (8.3) | 41.2 | (9.2) | |
| Male | 17 | (29.8) | 42 | (35.6) | 19 | (32.8) | 19 | (34.5) | |
| BMI | 48.9 | (6.9) | 49.3 | (5.0) | 49.4 | (6.0) | 50.4 | (6.3) | |
| Steatosis | 57/0/0/0 | (100/0/0/0) | 0/118/0/0 | (0/100/0/0) | 0/0/58/0 | (0/0/100/0) | 0/0/0/55 | (0/0/0/100) | ? |
| (0/1/2/3) | | | | | | | | | |
| NASH | 0 | (0) | 14 | (11.8) | 26 | (44.8) | 35 | (63.6) | ? |
| Fibrosis* | 55/2 | (97/3) | 95/23 | (81/19) | 48/10 | (83/17) | 32/22 | (58/40) | ? |
| (0-1/2-4) | | | | | | | | | |
| PNPLA3 | 40/13/2 | (73/24/4) | 64/44/9 | (54/37/8) | 26/24/5 | (45/41/5) | 12/36/7 | (22/65/13) | <10⁻⁶ |
| (CC/CG/GG) | | | | | | | | | |
| IGT | 8 | (14.0) | 24 | (20.3) | 19 | (32.8) | 21 | (38.2) | 0.007 |
| HbA1C | 0.055 | (0.004) | 0.056 | (0.004) | 0.057 | (0.004) | 0.058 | (0.004) | 0.003 |
| Insulin* | 139 | (72) | 197 | (153) | 196 | (105) | 236 | (87) | 0.008 |
| FPG (mM) | 5.4 | (0.5) | 5.6 | (0.6) | 5.7 | (0.6) | 5.8 | (0.5) | 0.009 |
| BP meds | 18 | (31.5) | 38 | (32.2) | 25 | (43.1) | 12 | (21.8) | |
| Lipid meds | 8 | (14.0) | 13 | (11.0) | 9 | (15.5) | 5 | (9.1) | |
| Tot chol | 4.6 | (0.8) | 4.7 | (0.8) | 4.8 | (0.9) | 4.7 | (0.7) | |
| (mM) | | | | | | | | | |
| HDL (mM) | 1.32 | (0.34) | 1.24 | (0.26) | 1.24 | (0.29) | 1.28 | (0.46) | |
| LDL (mM) | 2.72 | (0.75) | 2.75 | (0.75) | 2.69 | (0.78) | 2.69 | (0.76) | |
| TGs (mM) | 1.2 | (0.51) | 1.5 | (0.60) | 1.9 | (0.89) | 1.8 | (0.85) | <0.001 |
| ALT (U/L) | 24.1 | (26.3) | 29.4 | (16.1) | 35.3 | (18.0) | 41.0 | (17.1) | <0.001 |
| AST (U/L) | 20.0 | (13.4) | 22.7 | (11.2) | 25.6 | (9.6) | 29.7 | (9.4) | <0.001 |
| GGT (U/L) | 24.9 | (25.5) | 28.6 | (14.5) | 46.6 | (55.8) | 41.1 | (19.7) | <0.001 |
| Bili direct | 2.3 | (1.0) | 2.5 | (1.0) | 2.4 | (1.2) | 2.3 | (0.9) | |
| (umol/L) | | | | | | | | | |
| Bili total | 8.1 | (4.7) | 8.1 | (3.2) | 7.9 | (3.9) | 7.8 | (2.8) | |
| (umol/L) | | | | | | | | | |

| | NASH Status | | | | |
|---|---|---|---|---|---|
| | No NASH | | NASH | | p-value |
| n | 202 | | 74 | | |
| Age | 40.8 | (9.8) | 42.2 | (8.9) | |
| Male | 68 | (33.7) | 25 | (33.8) | |
| BMI | 49.2 | (5.8) | 50.2 | (6.1) | |
| Steatosis | 55/100/31/16 | (27/49/15/8) | 0/14/25/35 | (0/19/34/47) | ? |
| (0/1/2/3) | | | | | |
| NASH | 0 | (0) | 74 | (100) | ? |
| Fibrosis* | 175/27 | (87/13) | 46/28 | (62/38) | ? |
| (0-1/2-4) | | | | | |
| PNPLA3 | 115/71/13 | (58/36/7) | 27/37/9 | (37/51/12) | 0.007 |
| (CC/CG/GG) | | | | | |
| IGT | 47 | (23.3) | 22 | (29.7) | |
| HbA1C | 0.056 | (0.004) | 0.057 | (0.003) | 0.04 |
| Insulin* | 182.9 | (132.7) | 223.6 | (76.8) | |
| FPG (mM) | 5.6 | (0.6) | 5.8 | (0.6) | 0.001 |
| BP meds | 63 | (31.2) | 29 | (39.2) | |
| Lipid meds | 29 | (14.4) | 6 | (8.1) | |
| Tot chol | 4.6 | (0.8) | 4.8 | (0.7) | |
| (mM) | | | | | |
| HDL (mM) | 1.25 | (0.27) | 1.26 | (0.39) | |
| LDL (mM) | 2.71 | (0.78) | 2.76 | (0.72) | |
| TGs (mM) | 1.49 | (0.68) | 1.80 | (0.76) | 0.001 |
| ALT (U/L) | 28.6 | (19.9) | 40.8 | (17.7) | <0.001 |
| AST (U/L) | 22.2 | (11.5) | 29.0 | (9.4) | <0.001 |
| GGT (U/L) | 30.1 | (22.9) | 44.8 | (46.7) | 0.001 |
| Bili direct | 2.4 | (1.0) | 2.4 | (0.9) | |
| (umol/L) | | | | | |
| Bili total | 7.9 | (3.7) | 8.3 | (3.2) | |
| (umol/L) | | | | | |

This study was conducted according to the principles outlined in the Declaration of Helsinki and approved by the Institutional Review Boards at Université Laval and Duke University. Each participant provided written informed consent before participation.

Genotyping: PNPLA3 genotyping (r5738409) was performed on genomic DNA extracted from the blood buffy coat using the GenElute Blood Genomic DNA kit (Sigma, St. Louis, Mo., USA). Rs738409 was genotyped using validated primers and TaqMan probes (Applied Biosystems). PNPLA3 genotypes were determined using 7500 Fast Real-Time PCR System (Applied Biosystems) and analyzed using a high-throughput array technology QuantStudio 12K Flex system, coupled with Taqman OpenArray technology (Life Technologies).

Metabolite profiling: BCKA, amino acid, acylcarnitine, and ceramide profiles were derived from plasma. Briefly, plasma concentrations of the alpha-keto acids of leucine (α-keto-isocaproate, KIC), isoleucine (α-keto-β-methylvalerate, KMV) and valine (α-keto-isovalerate, KIV) were measured by liquid chromatography mass spectrometry (LC-MS) and amino acid, acylcarnitine, and ceramide profiling was performed by tandem mass spectrometry (MS/MS). All MS analyses employed stable-isotope-dilution with internal standards from Isotec, Cambridge Isotopes Laboratories, and CDN Isotopes. Methods of sample handling and extraction have been described previously (Ferrara et al., 2008; Newgard et al., 2009; Ronnebaum et al., 2006). All MS analyses employed stable-isotope-dilution with internal standards from Isotec, Cambridge Isotopes Laboratories, and CDN Isotopes.

Statistical analysis:All statistical analyses were carried out in R( ). For all tests a P-value <0.05 was considered significant. 80 metabolites (3 BCKA, 14 amino acids, 43 acylcarnitines, 20 ceramides) out of the 84 (3 BCKA, 15 amino acids, 45 acylcarnitines, 21 ceramides) initially measured met quality control standards and were subsequently used to generate PCA factors. The 18 factors that had an eigenvalue >1 and explained 73% of total variance were used for subsequent analysis alongside the three individual BCKA (KIV, KIC, KMV), BCAA (Val, Ile/Leu), and the BCKA to BCAA ratio (sum of KIV, KIC, KMV/sum of Val, Ile/Leu). The 18 PCA factors are displayed in Table 2.

TABLE 2
PCA factors.

| PCA Factor | Metabolite Components |
|---|---|
| Factor 1 - Even chain acylcarnitines | C14:1, C12:1, C2, C16:1, C14:2, C4-OH, |
| | C16:2, C14:1-OH, C12, C18:1, C6-DC/C8-OH |
| | C16:1-OH/C14:1-DC, C14, C12-OH/C10-DC, |
| | C18:1-OH/C16:1-DC, C16, C10:1, C10, C14- |
| | OH/C12-DC, C18:2, C8, C8:1-DC, C18:1~DC |
| Factor 2 - Glucosylceramides | d18:1/C24_cer. d18:1/C16_cer, |
| | d18:1/C24:1_cer, d18:1/C22_cer, |
| | d18:1/C23_cer, d18:1/C18_cer, |
| | d18:1/C20_cer, C16_cer |
| Factor 3 - Amino acid related | Phenylalanine, leucine/isoleucine, methionine, |
| | arginine, tyrosine, valine, omitihine, C3 AC, |
| | proline, citrulline, KMV, histidine |
| Factor 4 - Ceramides | C26:1_cer, C20_cer, C26_cer, C25_cer, |
| | d18:1/C26_cer, C18_cer |
| Factor 5 - Branched-chain keto and amino acids | KIC, KMV, KIV, leucine/isoleucine, valine |
| Factor 6 - Medium chain OH/DC acylcarnitines | C8:1-OH/C6:1-DC, C10-OH/C8-DC, C8:1-DC |
| Factor 7 - Glycine related amino acids | Glycine, serine, histidine |
| Factor 8 - C18/C16 acylcarnitines | C18, C16, C18:2, C18:1 |
| Factor 9 - Long chain ceramides | C24:1_cer, C16_cer, C20:1_cer, C18_cer |
| Factor 10 - Long chain acylcarnitines | C20:4, C22, C18;2-OH |
| Factor 11 - Medium chain unsaturated acylcarnitines | C8:1, C10:3, C10:2 |
| Factor 12 - Long chain ceramides | C23_cer, C22_cer, C24_cer |
| Factor 13 - Hydroxyisovaleryl/malonyl carnitine | C5-OH/C3-DC |
| Factor 14 - Alanine, proline | Alanine, proline |
| Factor 15- C20 acylcarnitine | C20 |
| Factor 16 - Short chain acylcarnitines | C4/Ci4, C3, C5 |
| Factor 17 - Long chain dicarboxyl acylcarnitines | C20-OH/C18-DC, C18:1-DC |
| Factor 18 - Medium chain acylcarnitines | C8, C10, C10:1 |

Proportional odds logistic regression was employed to test the association of the aforementioned variables with steatosis grade (Grade 0 (n=57), Grade 1 (n=118), Grade 2 (n=58), Grade 3 (n=55)). Logistic regression was used to test the metabolite/PCA factor associations with NASH (Yes (n=74)/NO (n=202)) and Fibrosis (Grade 0-1 (n=57) vs Grade 2-4 (n=230)). All associations were tested in both univariate and multivariate models and underwent Bonferroni correction for multiple comparisons. The multivariate model included metabolite/PCA factor plus HbA1c, ALT, AST, GGT, BMI, sex, age, rs738409 genotype, and study phase. Metabolites/factors that were found to have a significant association with the outcomes were tested for interactions with sex or genotype.

Results

Characteristics of the study population: The study population from the QHLI biobank was stratified by steatosis grade or NASH status among subjects otherwise closely matched for age, gender, and BMI (Table 1). A strong correlation was observed between steatosis grade and the presence of NASH and fibrosis. Subjects were genotyped for the presence of the PNPLA3 Ile148Met variant, which has been shown to be commonly associated with elevated liver fat in Mexican-American subjects. Although found in fewer individuals in the French-Canadian subjects represented in the QHLI biobank, the Ile148Met variant was clearly correlated with steatosis grade. Steatosis grade was also correlated with impaired glucose tolerance (IGT), HbA1c, insulin, and fasting plasma glucose (FPG) levels. There was no association between steatosis grade and the proportion of individuals taking medications for blood pressure (BP) or lipids (Table 1). Steatosis grade was also not associated with total cholesterol, high-density lipoprotein (HDL), or low-density liporotein (LDL) concentrations but was strongly associated with plasma triglycerides and circulating liver enzyme levels (ALT, AST, and GGT). Bilirubin levels were not associated with steatosis grade (Table 1).

Compared to individuals without NASH, presence of NASH was associated with more severe steatosis and a higher proportion of individuals with advanced fibrosis (grade 2-4 vs 0-1). The PNPLA3 Ile148Met variant, Hba1c, and FPG were also strongly associated with the presence of NASH. However, in contrast to steatosis grade, insulin levels and IGT were not associated with NASH. Associations between NASH and plasma lipids, liver enzymes, medications, and bilirubin mirrored those for steatosis grade (Table 1).

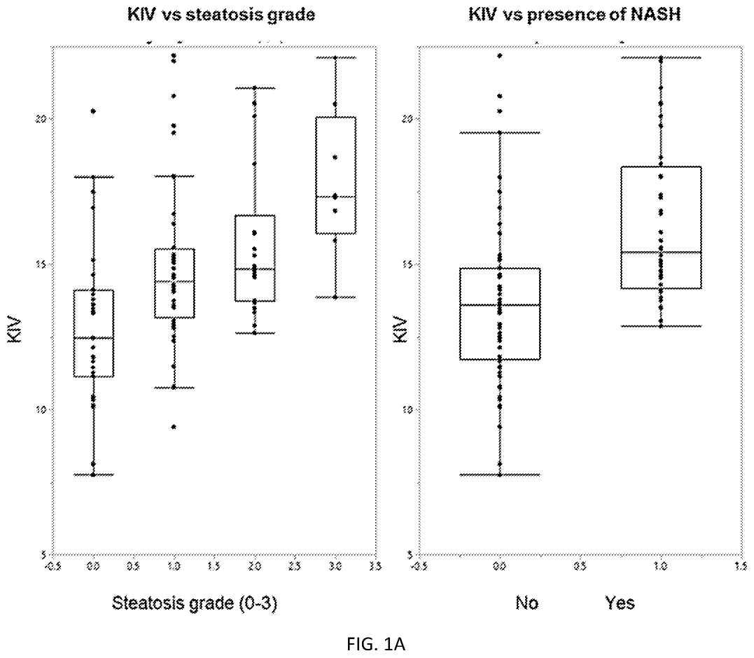

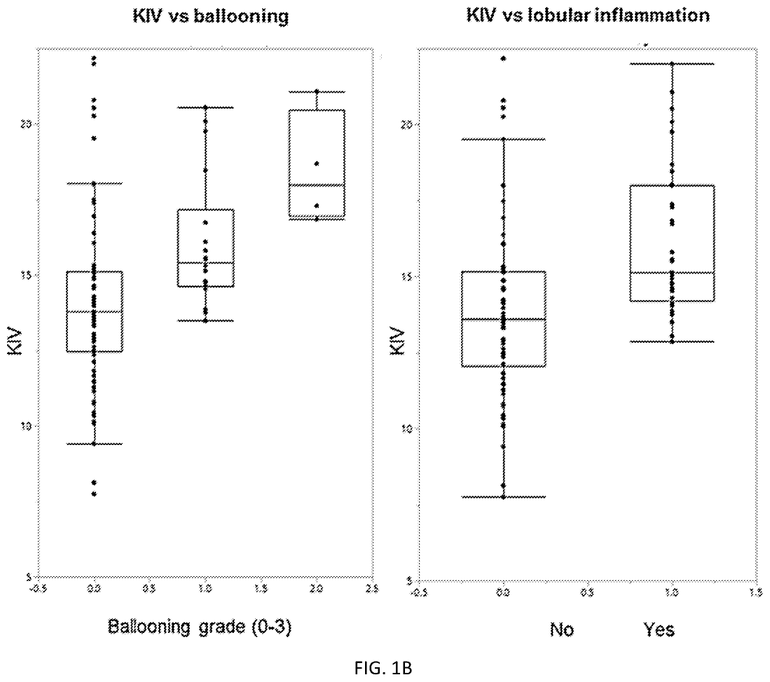

A BCKA-related signature of NAFLD status: Strong associations were observed between KIV, (the BCKA derived from valine), and the ratio of the molar sum of the branched chain keto acids:molar sum of branched chain amino acids (BCKA:BCAA ratio) with both steatosis grade and NASH (FIG. 1). Importantly, this cannot be simply explained by elevated BCAA supply since none of the individual BCAAs or the PCA factor 5, which is comprised of the three BCKA and their cognate BCAA, displayed an association with steatosis grade or NASH.

Remarkably, besides KIV and the BCKA:BCAA ratio, no other metabolite or metabolite factor was found to associate with both steatosis grade and NASH. Moreover, of the 18 PCA factors listed in Table 2, only Factor 14 (alanine and proline) and Factor 7 (glycine, serine, and histidine), were associated with steatosis grade, whereas, Factor 10 (C20:4, C22, C18:2-OH acylcarnitines) was the only metabolite factor associated with NASH (Table 3). No metabolites or factors measured in this study were found to associate with the presence of advanced fibrosis.

TABLE 3
Association of metabolites with NAFLD phenotypes.

| | | | Univariate model | | | Full model | | |
|---|---|---|---|---|---|---|---|---|
| Phenotype | Metabolite | n | OR | (95% CI) | pval | OR | (95% CI) | pval |
| | Factor14 | 288 | 0.65 | (0.53-0.81) | 1.3 × 10⁻⁴ | 0.6 | (0.47-0.77) | 3.6 × 10⁻⁵ |
| Steatosis grade | Factor7 | 288 | 3.71 | (1.37-2.15) | 2.5 × 10⁻⁶ | 1.68 | (1.33-2.16) | 2.6 × 10⁻⁵ |
| | KIV | 288 | 3.15 | (1.08-1.25) | 1.1 × 10⁻⁴ | 1.15 | (1.08-1.26) | 8.2 × 10⁻⁴ |
| | BCKA/SCAA | 288 | 1.49 | (1.2-1.85) | 2.9 × 10⁻⁴ | 1.34 | (1.06-1.7) | n.s. |
| | BCKA/BCAA | 276 | 2.01 | (1.51-2.71) | 3.0 × 10⁻⁶ | 2.15 | (1.54-3.06) | 1.2 × 10⁻⁵ |
| NASH | KIV | 276 | 1.19 | (1.08-1.31) | 4.0 × 10⁻⁴ | 1.27 | (1.13-1.44) | 1.1 × 10⁻⁴ |
| | Factor10 | 276 | 1.63 | (1.22-2.23) | 1.4 × 10⁻³ | 1.75 | (1.22-2.56) | n.s. |

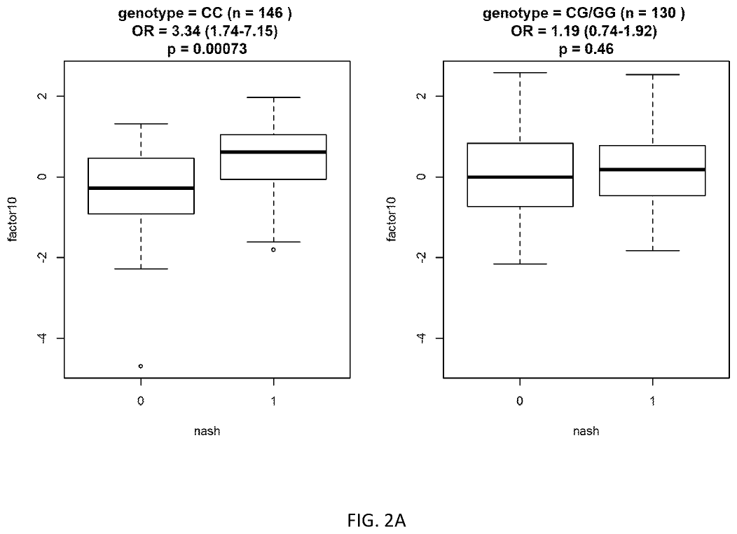

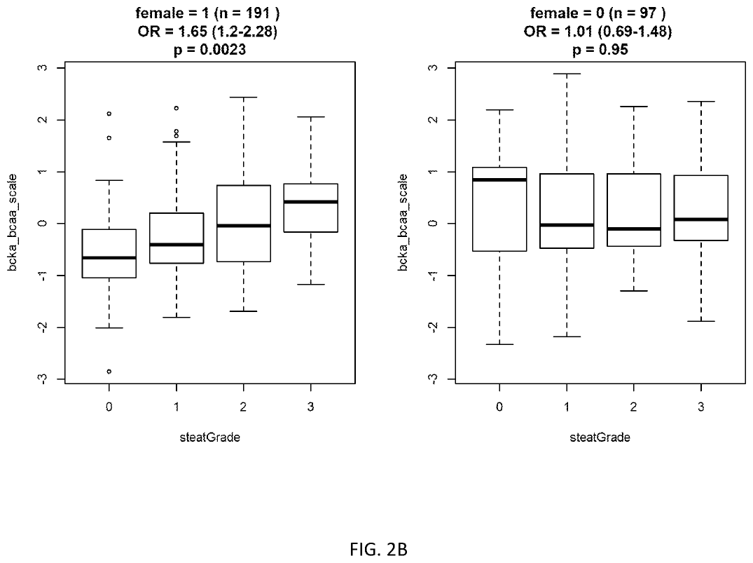

The associations of KIV with steatosis grade and NASH, and the BCKA:BCAA ratio with NASH were maintained when a multivariate statistical model that considered sex, yy, zz, etc, was applied. However, the association of BCKA:BCAA ratio with steatosis grade and Factor 10 with NASH were lost. It was therefore tested whether this could be due to sex or genotype effects. The association of the BCKA:BCAA ratio with steatosis grade is driven by females and completely absent in the male cohort (FIG. 2); whereas the association of Factor 10 with NASH was present in carriers of the major allele for PNPLA3 but absent in carriers of the PNPLA3 Ile148Met variant (FIG. 2).

### DISCUSSION

The comprehensive metabolic characterization of plasma from a well-matched population of persons with severe obesity revealed that the BCKA, KIV, and the BCKA:BCAA ratio were the only metabolite features strongly associated with both steatosis grade and NASH. This data also suggests that plasma levels of KIV or the BCKA/BCAA ratio could be used as a biomarker for NAFLD/NASH that might be leveraged in the clinic to identify individuals that might be best suited to therapies that target the lipogenic machinery.

The results described herein suggest that BCKAs, and not simply the BCAAs themselves, associate with NAFLD and NASH. This is likely due to the fact that circulating BCAA are raised by a diverse set of metabolic adaptations in the obese milieu. These include onset of insulin resistance causing impairment of incorporation of amino acids for protein synthesis, dietary changes, altered gut microbiota, transcriptional downregulation of the entire BCAA catabolic pathway in the expanding adipose tissue, and increased BDK:PPM1K ratio leading to inhibition of hepatic BCKDH activity. Importantly, whereas the liver does not metabolize BCAA effectively due to very low levels of the branched chain amino acid aminotransferase (BCAT), its high levels of BCKDH expression make it one of the most active sites of BCKA catabolism. Thus whereas BCAA are not an optimal marker of hepatic BCKDH activity or the balance of BDK and PPM1K in liver, the data shown herein strongly support the notion that measurement of plasma BCKAs or the ratio of BCKA (reporting in larger part on hepatic BDK/PPM1K balance) to BCAA (which provides correction for systemic BCAA load) provides a sensitive index of the balance of BDK/PPM1K in the liver and NAFLD. Remarkably this true even in severely obese individuals and carriers of the PNPLA3 risk allele.

Interestingly, whereas the single BCKA, KIV, was strongly associated with both steatosis grade and NASH in the total subject cohort, and independently, in males and females, the association of the BCKA:BCAA ratio with steatosis grade was evident only in females. This sex interaction may be driven by differences in the partitioning of BCAA. Indeed, levels of BCAA and related metabolites are known to be higher in males than females in a cohort of overweight and obese adults or in pediatric subjects with a comparable BMI. Moreover, sex-dependent differences in the relationship of BCAA with fasting glucose and lipids have been described in early adolescents. One possible mechanism underlying these observations is that the female sex hormone estrogen promotes BCAA uptake by inducing the expression of the cell polarity protein LLGL7L2, which binds to and activates the large neutral amino acid transporter SLC7A5 at the cell surface. The findings presented herein may also suggest differences in regulation of the hepatic BDK:PPM1K ratio in males in females, although this remains to be investigated. Additional study is warranted to better understand the differential regulation of BCAA utilization across the sexes and whether this contributes to any differences in risk for NAFLD and/or other cardiometabolic diseases.

Beyond KIV and the BCKA:BCAA ratio two additional amino acid related factors that were associated with steatosis grade were identified, as well as a long chain acylcarnitine related factor that was associated with the presence of NASH in carriers of the major PNPLA3 allele. Factors 7 and 14, are made up of glycine related amino acids (glycine, serine, and histidine) and nitrogen handing metabolites (alanine and proline), respectively. Importantly, the negative association of glycine and serine with steatosis grade and BCAA levels as observed herein has also been observed in other cohorts. Factor 10, comprised of C20:4, C22, C18:2 carnitines, was found to associate with the presence of NASH. Given that the metabolite with strongest loading in this factor is arachidonyl (C20:4)-carnitine, it is tempting to speculate that the association between this factor and NASH is driven by arachidonic acid derived lipid mediators known to play a role in inflammation such as the leukotrienes and prostaglandins.

In conclusion, this study provided the first proof of principal in humans that plasma levels of BCKA or the BCKA/BCAA ratio associate with NAFLD status in obese individuals. This finding also provides support for the idea that disequilibrium in the hepatic balance of the BCKDH kinase and phosphatase, now understood to also play a role in regulation of the critical DNL enzyme ACL, plays an important role in the development of NAFLD in obese humans. The major strengths of this study include the use of a well matched severely obese population and the use of samples in which liver phenotypes were determined via gold-standard histological grading.

### REFERENCES

Ferrara, C. T., Wang, P., Neto, E. C., Stevens, R. D., Bain, J. R., Wenner, B. R., Ilkayeva, O. R., Keller, M. P., Blasiole, D. A., Kendziorski, C., et al. (2008). Genetic networks of liver metabolism revealed by integration of metabolic and transcriptional profiling. PLoS Genet. 4, e1000034.
Newgard, C. B., An, J., Bain, J. R., Muehlbauer, M. J., Stevens, R. D., Lien, L. F., Haqq, A. M., Shah, S. H., Arlotto, M., Slentz, C. A., et al. (2009). A branched-chain amino acid-related metabolic signature that differentiates obese and lean humans and contributes to insulin resistance. Cell Metab. 9, 311-326.
Ronnebaum, S. M., Ilkayeva, O., Burgess, S. C., Joseph, J. W., Lu, D., Stevens, R. D., Becker, T. C., Sherry, A. D., Newgard, C. B., and Jensen, M. V (2006). A pyruvate cycling pathway involving cytosolic NADP-dependent isocitrate dehydrogenase regulates glucose-stimulated insulin secretion. J. Biol. Chem. 281, 30593-30602.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of diagnosing and treating non-alcoholic fatty liver disease (NAFLD) in a subject, comprising:
a. Obtaining a sample from the subject;
b. Measuring a concentration of one or more branched chain keto-acids (BCKAs) in the sample;
c. Diagnosing the patient with NAFLD when the concentration of the one or more BCKAs is elevated compared to a reference value; and
d. Providing therapy to the subject diagnosed with NAFLD.

2. The method of claim 1, wherein the one or more branched chain keto-acids are selected from ketoisovalereric acid (KIV), ketoisocaproic acid (KIC), and ketomethylvaleric acid (KMV).

3. The method of any of the preceding claims, comprising measuring KIV in the sample and diagnosing the patient with NAFLD when the concentration of KIV is elevated compared to a reference value.

4. The method of any of the preceding claims, further comprising measuring a concentration of one or more branched chain amino acids (BCAAs) in the sample, calculating a ratio of one or more BCKAs:one or more BCAAs in the sample, and diagnosing the patient with NAFLD when the ratio of one or more BCKAs:one or more BCAAs in the sample is elevated compared to a reference value.

5. The method of claim 4, wherein the branched chain amino acids are selected from leucine, isoleucine, and valine.

6. A method of diagnosing and treating non-alcoholic fatty liver disease (NAFLD) in a subject, comprising:
a. Obtaining a sample from the subject;
b. Measuring a concentration of one or more branched chain keto-acids (BCKAs) in the sample;
c. Measuring a concentration of one or more branched chain amino acids (BCAAs) in the sample;
d. Determining a ratio of one or more BCKAs:one or more BCAAs in the sample;
e. Diagnosing the patient with NAFLD when the ratio of one or more BCKAs:one or more BCAAs is elevated compared to a reference value; and
f. Providing therapy to the subject diagnosed with NAFLD.

7. The method of claim 6, wherein the one or more branched chain keto-acids are selected from ketoisovalereric acid (KIV), ketoisocaproic acid (KIC), and ketomethylvaleric acid (KMV).

8. The method of claim 6 or 7, wherein the branched chain amino acids are selected from leucine, isoleucine, and valine.

9. A method of diagnosing and treating non-alcoholic fatty liver disease (NAFLD) in a subject, comprising:
a. Obtaining a sample from the subject;
b. Measuring a concentration of one or more branched chain keto-acids (BCKAs) in the sample;
c. Measuring a concentration of one or more branched chain amino acids (BCAAs) in the sample;
d. Determining a ratio of one or more BCKAs:one or more BCAAs in the sample;
e. Diagnosing the patient with NAFLD when the concentration of the one or more BCKAs and the ratio of the one or more BCKAs: the one or more BCAAs is elevated compared to a reference value; and
f. Providing therapy to the subject diagnosed with NAFLD.

10. The method of claim 9, wherein the one or more branched chain keto-acids are selected from ketoisovalereric acid (KIV), ketoisocaproic acid (KIC), and ketomethylvaleric acid (KMV).

11. The method of claim 9 or 10, wherein the branched chain keto-acid is KIV.

12. The method of any one of claims 9-11, wherein the branched chain amino acids are selected from leucine, isoleucine, and valine.

13. The method of any of the preceding claims, wherein the NAFLD is non-alcoholic steatohepatitis.

14. The method of any of the preceding claims, wherein the concentration of the one or more branched chain amino acids and/or the concentration of the one or more branched chain amino acids is measured by mass spectrometry.

15. The method of any of the preceding claims, wherein the sample is selected from blood, serum, and plasma.

16. The method of any of the preceding claims, wherein the subject is a human.

17. The method of any of the preceding claims, wherein the subject is overweight or obese.

18. The method of any of the preceding claims, wherein the subject is female.

19. The method of any of the preceding claims, wherein the subject expresses the Ile148Met variant of PNPLA3.

20. The method of any of the preceding claims, wherein the treatment comprises one or more therapeutic agents and/or surgery.

21. The method of claim 20, wherein one or more therapeutic agents are selected from antioxidants, cytoprotective agents, antidiabetic agents, insulin-sensitizing agents, anti-hyperlipidemic agents, acetyl co-A carboxylase inhibitors, and ATP-citrate lyase inhibitors.

22. A panel of biomarkers comprising one or more branched chain keto-acids (BCKAs) and one or more branched chain amino-acids (BCAAs).

23. The panel of claim 22, wherein the branched chain keto-acids are selected from alpha-ketoisovalereric acid (KIV), alpha-ketoisocaproic acid (KIC), and alpha-keto-beta-methylvaleric acid (KMV) and wherein the branched chain amino acids are selected from leucine, isoleucine, and valine.

24. The panel of any of the preceding claims for use in a method of diagnosing non-alcoholic fatty liver disease (NAFLD) in a subject, wherein an elevated concentration of one or more BCKAs in the panel compared to a reference value and/or an elevated ratio of one or more BCKAs:BCAAs in the panel compared to a reference value is positively correlated with NAFLD in the subject.

25. The panel of claim 24, wherein the NAFLD is non-alcoholic steatohepatitis.

26. The panel of 24 or 25, wherein concentration of the one or more biomarkers in the panel is measured by mass-spectrometry.

27. The panel of any one of claims 24-26, wherein the concentration of the one or more biomarkers in the panel is measured in a sample obtained from the subject.

28. The panel of claim 27, wherein the sample is selected from blood, serum, and plasma.

29. The panel of any one of claims 24-28, wherein the subject is a human.

30. The panel of any one of claims 24-29, wherein the subject is overweight or obese.

31. The panel of any one of claims 24-30, wherein the subject is female.

32. The panel of any one of claims 24-31, wherein the subject expresses the Ile148Met variant of PNPLA3.

33. A method comprising:
a. Obtaining a sample from the subject;
b. Measuring a concentration of one or more branched chain keto-acids (BCKAs) and one or more branched chain amino acids (BCAAs) in the sample; and
c. Calculating the ratio of one or more BCAAs:one or more BCAAs: in the sample.

34. The method of claim 33, wherein the branched chain keto-acids are selected from alpha-ketoisovalereric acid (KIV), alpha-ketoisocaproic acid (KIC), and alpha-keto-beta-methylvaleric acid (KMV) and wherein the branched chain amino acids are selected from leucine, isoleucine, and valine.

35. The method of claim 33 or 34, wherein the concentration is measured by mass spectrometry.

36. The method of any one of claims 33-35, wherein the sample is selected from blood, serum, and plasma.

37. The method of any one of claims 33-36, wherein an elevated concentration of the one or more BCKAs compared to a reference value and/or an elevated ratio of one or more BCKAs:one or more BCAAs in the sample is positively correlated with non-alcoholic fatty liver disease (NAFLD) in the subject.

38. The method of claim 37, wherein the NAFLD is non-alcoholic steatohepatitis.

39. The method of any one of claims 33-38, wherein the subject is a human.

40. The method of any one of claims 33-39, wherein the subject is overweight or obese.

41. The method of any one of claims 33-40, wherein the subject is female.

42. The method of any one of claims 33-41, wherein the subject expresses the Ile148Met variant of PNPLA3.
